(19) United States
(12) Patent Application Publication
(10) Pub. No.: US 2007/0243273 A1
(43) Pub. Date: Oct. 18, 2007

(54) Reduction of the Deleterious Effects of Tobacco Smoking

(75) Inventors: Inderjit Kumar Dev, Durham, NC (US); Ven Subbiah, Greenville, NC (US)

Correspondence Address: PASSE' INTELLECTUAL PROPERTY, LLC, 1717 BRASSFIELD RD., RALEIGH, NC, 27614, US

(73) Assignee: SaviPu Pharmaceuticals, Durham, NC (US)

(21) Appl. No.: 11/734,595

(22) Filed: Apr. 12, 2007

Related U.S. Application Data
(60) Provisional application No. 60/791,239, filed on Apr. 12, 2006.

(51) Int. Cl. A61K 36/81; A61K 31/12

(52) U.S. Cl. 424/751; 514/690

(57) ABSTRACT

The invention features a method for inhibiting or reducing the deleterious effects of tobacco smoking including increased incidence of cancers, cardiovascular diseases, various lung disorders and the oxidative effects. The method includes the incorporation of an effective amount of a compound or a standardized plant extract that is that comprises an inhibitor of NF-κB into tobacco or a tobacco product. In particular, it relates to useful NF-κB inhibitor diterpenes and compositions containing them for incorporation into tobacco or a tobacco product.

This application claims priority of U.S. application Ser. No. 60/791,239 filed on Apr. 12, 2006 and incorporated herein in its entirety by reference.

### BACKGROUND OF THE INVENTION

1. Field of the invention

The field of the invention relates to the treatment of tobacco to lesson the deleterious effect of tobacco smoking. In particular the invention relates to the treatment of tobacco or a tobacco product with an effective amount of a NF-κB inhibitor such that the deleterious effects of smoking tobacco are reduced.

2. Description of the Related Art

Tobacco smoke (TS) is a major risk factor for a number of diseases including cancer, chronic obstructive pulmonary disease (COPD) and cardiovascular disease. Smokers have a high incidence of lung cancer, which is the most common cause of cancer in Western countries, accounting for more deaths than those caused by prostrate, breast, and colorectal cancers combined. Recent estimates indicate that TS causes approximately 80-90% of lung cancer in the United States. TS is also implicated in cancers of the larynx, oral cavity, pharynx, esophagus, pancreas, kidney, and bladder. A study done by Zhang and Cai determined that heavy smokers have a higher incidence of chronic inflammation and lung impairment resulting in COPD, with ˜70% of COPD cases emanating from smokers. Besides COPD, inflammation is a cardinal feature of other lung diseases, including asthma, emphysema, sarcoidosis, and infectious diseases such as tuberculosis and Pneumocystis carinii pneumonia. TS is a complex chemical mixture containing thousands of different compounds, of which 100 are known carcinogens, co-carcinogens, mutagens, or tumor promoters. Metabolites of tobacco-specific N-nitrosamines, like 4-(methylnitrosamino)-1-(3-pyridyl)-1-butanone (NNK) and polycyclic aromatic hydrocarbons, like diolepoxides of benzo[a]pyrene (B[a]P) are believed to be the primary tobacco carcinogens. Tobacco smoke contains around 10¹⁷ oxidant molecules per puff, and this, together with a large body of evidence demonstrating increased oxidative stress in smokers contributes towards oxidant/antioxidant imbalance in the pathogenesis of above diseases. Moreover, active smokers have greater than 25% lower circulating concentrations of ascorbic acid, α-carotene, β-carotene, and cryptoxanthin. TS is also known to induce morphological changes in lungs, placenta, liver, and kidneys. Since it apparent that as long as tobacco is available people will continue to smoke, agents that can neutralize the effects of TS are urgently needed. A new concept indicates that chronic inflammation plays a crucial role in the development of certain cancers, cardiovascular diseases, and lung disorders. Although numerous different pathways are activated during the inflammatory response, one particular type of pathway is thought to be of paramount importance in inflammation is a pathway that involves NF-κB. NF-κB is a transcription factor that resides in the cytosol in an inactive form. The p50-p65 heterodimer is retained in the cytoplasm by the inhibitory subunit IB. On activation of the complex, IB sequentially undergoes phosphorylation, ubiquitination and degradation, thus releasing the p50-p65 heterodimer for translocation to the nucleus. An IB kinase, IKK, has been identified that phosphorylates serine residues in IB at position 32 and 36. Treatment of cells with tobacco smoke activates the IKK, thus leading to the degradation of IB and activation of the transcription factor. A wide variety of other agents including stress, viruses, bacteria, inflammatory stimuli, cytokines, free radicals, carcinogens, tumor promoters, and endotoxins also induce activation of NF-κB. On activation, NF-κB regulates the expression of almost 400 different genes, which include enzymes (e.g., COX-2, 5-LOX, and iNOS), cytokines (such as TNF, IL-1, IL-6, IL-8, and chemokines), adhesion molecules, cell cycle regulatory molecules, viral proteins, and angiogenic factors. The constitutive activation of NF-κB has been linked with a wide variety of human diseases, including asthma, atherosclerosis, AIDS, rheumatoid arthritis, COPD, diabetes, osteoporosis, Alzheimer's disease, and cancer. Several agents are known to suppress NF-κB activation, including Th2 cytokines (IL-4, IL-13, and IL-10), interferons, endocrine hormones (LH, HCG, MSH, and GH), phytochemicals, corticosteroids, and immunosuppressive agents. NF-κB has long been recognized as a signal indicator of problems in the body.

Smoking is a noxious process that also triggers oxidative stress, not only in first-hand smokers but also in those exposed to second-hand smoke. Reactive oxygen species (ROS) are produced in all mammalian cells from as a result of ongoing chermica events, such as oxidative phospliorylation, uric acid metabolism and prostagiandin synthesis. Cellular defense mechanisms have evolved to protect cells from ROS, and these include repair systems, detoxifying enzymes such as superoxide dismutases (SODs), and small molecule scavengers such as glutathione. An imbalance between the mechanisms that generate and protect against ROS results in compensatory oxidative stress or even oxidative damage, including DNA damage. In addition to these deleterious effects, ROS appear to have necessary signaling functions in the modulation of apoptosis, stress and proliferative signaling pathways. These observations suggest that ROS may be an important target for cancer chemoprevention. Consistent with this notion, mouse knock-outs of prdx1, the gene encoding a ROS scavenger and antioxidant protein peroxiredoxin 1, display susceptibility to tumors. Many antioxidants such as vitamins E, carotenoids and selenium have shown significant cancer chemo-preventive activity in animal models and humans.

An imbalance between oxidants and antioxidants is also considered to play a role in the pathogenesis of COPD. There is considerable evidence that an increased oxidative burden occurs in the lungs of patients with this disorder, and this may be involved in many of the pathogenic processes, such as direct injury to lung cells, mucus hypersecretion, inactivation of antiproteases, and enhancing lung inflammation through activation of redox-sensitive transcription factors. COPD is now recognized to have multiple systemic consequences, such as weight loss and skeletal muscle dysfunction. Moreover, it is appreciated that oxidative stress extends beyond the lung and may, through similar oxidative stress mechanisms as those in the lung, contribute to several of the systemic manifestations in COPD.

Oxidative stress also plays a role in enhancing inflammation through the upregulation of redox-sensitive transcription factors, such as NF-κB and activating protein 1 (AP-1), and also the extracellular signal-regulated kinase (ERK), c-Jun N-terminal kinase (JNK), and p38 mitogen-activated protein kinase pathways. Tobacco smoke has been shown to activate all of these signaling mechanisms. Studies in macrophage cell lines and alveolar and bronchial epithelial cells show that oxidants cause the release of inflammatory mediators, such as IL-8, IL-1, and NO, and that these events are associated with increased expression of the genes for these inflammatory mediators and increased nuclear binding or activation of NF-κB. The linking of NF-κB to its consensus site in the nucleus leads to enhanced transcription of proinflammatory genes and therefore inflammation, which itself will produce more oxidative stress, creating a vicious circle of enhanced inflammation resulting from the increased oxidative stress. Animal models of smoke exposure show that neutrophil influx in the lungs is associated with increased IL-8 gene expression and protein release and with NF-κB activation. All of these events are associated with oxidative stress because they can be abrogated by antioxidant therapy.

To date there are no products which are included in tobacco products which are shown to be capable of blocking, reducing or inhibiting the deleterious effects of smoking tobacco during the smoking process.

Accordingly, it would be extremely useful to find compositions that block both the generation of reactive oxygen species and the activation of NF-κB induced by tobacco smoke and that could be included with tobacco or a tobacco containing product and retain its effectiveness .

### SUMMARY OF THE INVENTION

It has been discovered that smoking tobacco or a tobacco containing product containing or treated with a NF-κB inhibitor acts to inhibit or reduce the deleterious effects in a human or other mammal caused by the smoking of tobacco when compared to untreated tobacco.

One aspect of the invention includes tobacco or a tobacco containing product containing or having applied thereto an effective amount of a NF-κB inhibitor sufficient to prevent or reduce the deleterious effects on a mammal smoking the tobacco or tobacco product.

Another aspect of the invention includes a method for reducing the deleterious effects induced by tobacco smoking comprising incorporating an effective amount of a composition containing a NF-κB inhibitor into tobacco or a tobacco containing product prior to smoking the tobacco or tobacco product.

Other embodiments will be clear from the disclosure herein and the disclosure of this invention which includes the novel concept of including a NF-κB inhibitor in a tobacco containing product.

### DETAILED DESCRIPTION OF THE INVENTION

To date several NF-κB inhibitors have been described in the literature and shown to be useful in vivo. It is clear that those skilled in the art would be able to incorporate many of those compositions into the present invention. It is also clear that the exact amount of compound can be determined on a compound by compound basis and now that it is clear that compounds are not entirely destroyed in the smoking process tailor an appropriate dosage and compound selection.

Tanshinone IIA is a purified component of ‘Danshen’, an important traditional Chinese medicine used for treating many diseases, especially ischemic cardiovascular diseases. Tanshinone IIA can be isolated from clary sage (Salvia sclarea L.) and other Salvia species as disclosed in U.S. Application # 20050008710, and application Attorney docket number SVP004 and filed concurrently herewith both of which hereby incorporated by reference.

Tanshinone IIA has antioxidant properties that protect against lipid peroxidation and it has also demonstrated inhibition of NF-κB as disclosed in U.S. Application Ser. No. 60/779,142, hereby incorporated by reference. Since generation of reactive oxygen species and activation of NF-κB leads to cancers, cardiovascular diseases, and lung disorders, antioxidant compounds that inhibit the activation or action of NF-,B is effective in neutralizing the deleterious effects of tobacco smoke.

Metabolites of tobacco-specific N-nitrosamines, like 4-(methylnitrosamino)-1-(3-pyridyl)-1-butanone (NNK) and polycyclic aromatic hydrocarbons, like diolepoxides of benzo[a]pyrene are believed to be the primary tobacco carcinogens (Hecht, S. S. (1999) Tobacco smoke carcinogens and lung cancer. J. Natl Cancer Inst., 91, 1194-1210). Tanshinone IIA at very low concentrations (3 nM) totally prevented benzo[a]pyrene-induced transformation of rat tracheal epithelial cells. These concentrations of Tanshinone IIA were nontoxic to the cells.

Accordingly, the invention features a method for reducing the deleterious effects of cigarette smoking by incorporating an effective amount of a compound or a standardized plant extract that is an antioxidant and or inhibitor of NF-κB into cigarettes.

Since tanshinone IIA has shown efficacy in neutralizing the deleterious effects of NF-κB with minimal cytotoxicity, other tanshinones, e.g., as disclosed in U.S. Application Ser. No. 60/779,142, and hereby incorporated by reference, may show similar or greater efficacy.

Additional compounds that are known to inhibit NF-κB include, without limitation, .alpha.-lipoic acid (Sen et al., 1998; Suzuki et al., 1992), alpha.-tocopherol (Islam et al., 1998), Anetholdithiolthione (ADT) (Sen et al., 1996), Butylated hydroxyanisole (BHA) (Israel et al., 1992; Schulze-Osthoff et al., 1993), Cepharanthine (Okamoto et al., 1994), Caffeic Acid Phenethyl Ester (3,4-dihydroxycinnamic acid, CAPE) (Natarajan et al., 1996), Catechol Derivatives (Suzuki et al., 1994), Diethyldithiocarbamate (DDC) (Schreck et al., 1992b), Deferoxamine (Sappey et al., 1995), Dihydrolipoic Acid (Suzuki et al., 1995), Disulfiram (Schreck et al., 1992b), Dimethyldithiocarbamates (DMDTC) (Pyatt et al., 1998a), Curcumin (Diferulolylmethane) (Singh and Aggarwal, 1995b), Ebselen (Schreck et al., 1992b), EPC-K1 (phosphodiester compound of vitamin E and vitamin C) (Hirano et al., 1998) Epigallocatechin-3-gall-ate (EGCG; green tea polyphenols) (Lin et al., 1997; Yang et al., 1998), Ethylene Glycol Tetraacetic Acid (EGTA) (Janssen et al., 1999), Gamma-glutamylcysteine synthetase (gamma-GTS) (Manna et al., 1999), Glutathione (Cho et al., 1998; Schreck et al., 1992b), L-cysteine (Mihm et al., 1991) Lacidipine (Cominacini et al., 1998), Manganese Superoxide Dismutase (Mn-SOD) (Manna et al., 1998), Melatonin (Gilad et al., 1998; Mohan et al., 1995), N-acetyl-L-cysteine (NAC) (Schreck et al., 1991), Nordihydroguaiaritic acid (NDGA) (Brennan et al., 1998; Isral et al., 1992; Schulze-Osthoffet al., 1993; Staal et al., 1993), Orthophenanthroline (Schreck et al., 1992b), Phenylarsine oxide (PAO, tyrosine phosphatase inhibitor) (Arbault et al., 1997), Pyrrolidinedithiocarbamate (PDTC) (Schreck et al., 1992a), Quercetin (Musonda and Chipman, 1998), Rotenone (Schulze-Osthoff et al., 1993), S-allyl-cysteine (SAC, garlic compound) (Geng et al., 1997), Tepoxalin (5-(4-chlorophenyl)-N-hydroxy-(4-methoxyphenyl)-N-methyl-1H-pyrazole-3-propanamide) (Kazmi et al., 1995), Vitamin C (Staal et al., 1993), Vitamin E derivatives (Suzuki and Packer, 1993a), .alpha.-torphryl succinate (Staal et al., 1993; Suzuki and Packer, 1993b), .alpha.-torphryl acetate (Suzuki et al., 1993a), PMC (2,2,5,7,8-pentamethyl-6-hydroxychromane) (Suzuki et al., 1993a), Peptide Aldehydes: ALLnL (N-acetyl-leucinyl-leucinyl-norleucinal, MG101), LLM (N-acetyl-leucinyl-leucinyl-methional), Z-LLnV (carbobenzoxyl-leucinyl-le-ucinyl-norvalinal, MG1 15), Z-LLL (carbobenzoxyl-leucinyl-leucinyl-leucina-l, MG132) (Palombella et al., 1994; Grisham et al., 1999; Jobin et al., 1998a), Lactacystin, .beta.-lactone (Fenteany et al., 1998; Grisham et al., 1999), Boronic Acid Peptide (Grisham et al., 1999; Iqbal et al., 1995), Ubiquitin Ligase Inhibitors (Yaaron et al., 1997), Cyclosporin A (Frantz et al., 1994; Marienfield et al., 1997; McCaffrey et al. 1994; Meyer et al., 1997; Wechsler et al., 1994), FK506 (Tacrolimus) (Okamoto et al., 1994; Venkataraman et al., 1995), Deoxyspergualin (Tepper et al., 1995), APNE (N-acetyl-DL-phenylalanine-.beta.-naphthylester) (Higuchi et al., 1995), BTEE (N-benzoyl L-tyrosine-ethylester) (Rossi et al., 1998), DCIC (3,4-dichloroisocoumarin), DFP (diisopropyl fluorophosphate), TPCK (N-60-tosyl-L-phenylalanine chloromethyl ketone), TLCK (N-.alpha.-tosyl-L-lysine chloromethyl ketone) (D'Acquisto et al., 1998), Aspirin, sodium salicylate (Frantz and O'Neill, 1995; Kopp and Ghosh, 1994; Yin et al., 1998), BAY-117821 (E3((4-2-prop-enenitrile), BAY-117083 (E3((4-t-butylphenyl)-2-propenenitrile), Cycloepoxydon, 1-hydroxy-2-hydroxymethyl-3-pent-1-enylbenzene (Gehrt et al., 1998), Extensively oxidized low density lipoprotein (ox-LDL), 4-Hydroxynonenal (HNE) (Brand et al., 1997; Page et al., 1999), Ibuprofen (Palayoor et al., 1999), Nitric Oxide (NO) (Katsuyama et al., 1998; Matthews et al., 1996), Prostaglandin Al (Rossi et al., 2000), Sanguinarine (pseudochelerythrine, 13-methyl-[1,3]-benzodioxolo-[5,6-c]-1-,3-dioxolo-4,5 phenanthridinium) (Chaturvedi et al., 1997), Sulfasalazine (Wahl et al., 1998), Sulindac (Yamamato et al., 1999), YopJ (encoded by Yersinia pseudotuberculosis) (Schesser et al., 1998), .alpha.-melanocyte-stimulating hormone (.alpha.-MSH) (Manna and Aggarwal, 1998a), .beta.-lapachone (Manna et al., 1999a), Capsaicin (8-methyl-N-vanillyl-6-nonenamide) (Singh et al., 1996b), Core Protein of Hepatitis C virus (HCV) (Shrivastava et al., 1998), Diamide (tyrosine phosphatase inhibitor) (Toledano and Leonard, 1991; Singh and Aggarwal, 1995a), Emodin (3-methyl-1,6,8-trihydroxyanthraquinone) (Kumar et al., 1998), Erbstatin (tyrosine kinase inhibitor) (Natarajan et al., 1998), Estrogen (E2) (Sun et al., 1998), Fungal gliotoxin (Pahl et al., 1996), Genistein (tyrosine kinase inhibitor) (Natarajan et al., 1998), IL-13 (Manna and Aggarwal, 1998b), Leflunomide metabolite (A77 1726) (Manna and Aggarwal, 1999), Pervanadate (tyrosine phosphatase inhibitor) (Singh and Aggarwal, 1995a; Singh et al., 1996a), Phenylarsine oxide (PAO, tyrosine phosphatase inhibitor) (Mahboubi et al., 1998, Singh and Aggarwal, 1995a), Resiniferatoxin (Singh et al., 1996), Sesquiterpene lactones (parthenolide) (Hehner et al., 1998), beta.-amyloid protein (Bales et al., 1998), Glucocorticoids (dexametasone, prednisone, methylprednisolone) (Auphan et al., 1995; Brostjan et al., 1996; Ray and Prefontaine, 1994; Scheinman et al., 1995), IL-10 (Ehrlich et al., 1998; Lentsch et al., 1997), IL-11 (Trepicchio and Dorner, 1998), Leptomycin B (LMB) (Rodriguez et al., 1999), NLS Cell permeable peptides (Lin et al., 1995), o,o′-bismyristoyl thiamine disulfide (BMT) (Shoji et al., 1998), ADP ribosylation inhibitors (nicotinamide, 3-aminobenzamide) (Le Page et al., 1998), Atrial Natriuretic Peptide (ANP) (Gerbes et al., 1998), Atrovastat (HMG-COA reductase inhibitor) (Bustos et al., 1998; Hemandez-Presa et al., 1998), Calcitriol (1a,25-dihydroxyvitamine D3) (Harant et al., 1998), Clarithromycin (Miyanohara et al., 2000), Diamide (Toledano and Leonard, 1991), E3330 (quinone derivative) (Hiramoto et al., 1998), Glycyrrhizin (Wang et al., 1998), Herbimycin A (Iwasaki et al., 1992; Mahon and O'Neill,1995), Hypericin (Bork et al., 1999), Hydroquinone (HQ) (Pyatt et al., 1998b), IL-4 (Manna and Aggarwal 1999), I.kappa.B-like proteins (encoded by ASFV) (Powell et al., 1996; Revilla et al., 1998), KT-90 (morphine synthetic derivative) (Sueoka et al., 1998), Metals (chromium, cadmium, gold, mercury, zinc, arsenic) (Shumilla et al., 1998; Yang et al., 1995), Mevinolin, 5′-methylthioadenosine (MTA) (Law et al., 1992), N-ethyl-maleimide (NEM) (Toledano and Leonard, 1991), Nicotine (Sugano et al., 1998), Pentoxifylline (1-(5′-oxohexyl) 3,7-dimetylxanthine, PTX) (Biswas et al., 1993; Wang et al., 1997), Phenyl-N-tert-butylnitrone (PBN) (Kotake et al., 1998), Pituitary adenylate cyclase-activating polypeptide (PACAP) (Delgado et al., 1998), Pyrithione ( Kim et al., 1999), Quinadril (ACE inhibitor) (Bustos et al., 1998; Hernandez-Presa et al., 1998), Ribavirin (Fiedler et al., 1996), Secretory leukocyte protease inhibitor (SLPI) (Jin et al., 1997), Serotonin derivatives (N-(p-coumaroyl) serotonin, SC) (Kawashima et al., 1998), Silymarin (Saliou et al., 1998), Vascular endothelial growth factor (VEGF) (Oyama et al., 1998; Gabrilovich et al., 1998), Vasoactive intestinal peptide (VIP) (Delgado et al., 1998), D609 (phosphatidylcholine-phospholipase C inhibitor) (Bergmann et al., 1998), R031-8220 (PKC inhibitor) (Bergmann et al., 1998), SB203580 (p38 MAPK inhibitor) (Bergmann et al., 1998), Triptolide (PG490, extract of Chinese herb) (Qiu et al., 1999), LY294,002 (Sizemore et al., 1999), Mesalamine (Egan et al., 1999), Wortmannin (fungal metabolite) (Manna and Aggarwal, 2000), lactacystin, idoxifene, raloxifene, droloxifene, tiremifene, and tamoxifen. Further examples of compounds that inhibit NF-κB are disclosed in Narayanan et al. (U.S. Pat. No. 5,591,840), Bennett et al. (U.S. Pat. No. 6,069,008), Lai et al. (U.S. Pat. No. 6,316,502), Morishita et al. (U.S. Pat. No. 6,262,033), Qabar et al. (U.S. Pat. No. 6,117,896), and lino et al. (U.S. Pub. No. 2001/018441), each of which is hereby incorporated by reference.

Flavinoids, e.g., those found in soybean (such as genestein), can also be used to attenuate the deleterious effects of TS according to the invention. Among other possible flavinoids that can be used in the invention are galloyl flavonol glycosides such as quercetin or kaempferol.

Selenium (antioxidant; van den Brandt PA, Goldbohm RA, van't Veer P et al. Prospective cohort study on selenium status and the risk of lung cancer. Cancer Res 1993; 53: 4860-4865) and deguelin (AKT kinase inhibitor; Udeani GO et al. Cancer chemopreventive activity mediated by deguelin, a naturally occurring rotenoid Cancer Res 1997; 57: 3424-3428) can also be used to attenuate the deleterious effects of TS according to the invention.

In one embodiment of the invention the NF-κB inhibitor is selected from the group consisting of resveratrol, lactacystin, epigallocatechin, curcumin, pyrrolidine dithiocarbamate, herbamycin A, selenium, deguelin, idoxifene, raloxifene, droloxifene, tiremifene, and tamoxifen.

Incorporation of the NF-κB inhibitor into tobacco or into a tobacco product is well within the skill in the art. The composition can be sprayed on tobacco the cigarette papers fillers or any other ingredient in the product. The NF-κB inhibitor can also be included as a separate ingredient that is merely mixed into the product. Those skilled in the art will be able to produce products that meet the criteria of inclusion without further teaching herein.

### EXAMPLE 1

The rat tracheal epithelial (RTE) cell focus inhibition assay was used to identify potential chemopreventive activity of tanshinone IIA. Tanshinone IIA was evaluated for its ability to inhibit benzo[a]pyrene-induced transformation of RTE cells. Tracheal epithelial cells were isolated from 8-12 week-old male Fisher 344 rats and plated on collagen coated dishes. Initially, a nontoxic concentration of tanshinone IIA was determined by treating RTE cells with a range of 1.5 nM to 3000 nM of tanshinone IIA. Tanshinone IIA at concentrations of 150 nM and below was found to be nontoxic to RTE cells. About 10% cells survived at a concentration of 1500 nM. Freshly isolated RTE cells were exposed to benzo[a]pyrene alone or in combination with tanshinone IIA. Benzo[a]pyrene was washed out after an exposure of 24 h. The maximal non-toxic concentration and four half log concentrations of tanshinone IIA (300 nM to 3 nM) were used for the chemopreventive assay. After 30 days in culture, transformed foci were scored and inhibition was quantitated. A complete inhibition of transformation frequency by tanshinone IIA at each dose level was observed. A positive control of retinoic acid inhibited the transformation frequency by 50% at 1 nM.

### EXAMPLE 2

The same experiment is repeated except the administered dosage of NF-κB inhibitor is delivered via the NF-κB inhibitor applied to a tobacco sample which is in tern burned in the presence of cells. The results of the experiment are similar to example one and indicate this method of administration is effective to deliver an effective amount of a NF-κB inhibitor to a mammal.

The previous examples are not intended to be limiting. Various choices of NF-κB inhibitors, methods of application, tobacco products, dosages and the like are within the skill of one in the art and are therefore included in the claim invention which follows.

What is claimed is:

1. Tobacco or a tobacco containing product containing or having applied thereto an effective amount of a NF-κB inhibitor sufficient to prevent or reduce the deleterious effects on a mammal smoking the tobacco or tobacco product.

2. Tobacco or a tobacco containing product according to claim 1 wherein the NF-κB inhibitor is Tanshinone IIA or a diterpene derivative thereof which is a NF-κB inhibitor.

3. A method for reducing the deleterious effects induced by tobacco smoking comprising incorporating an effective amount of a composition containing a NF-κB inhibitor into tobacco or a tobacco containing product prior to smoking the tobacco or tobacco product.

4. A method according to claim 3 wherein the NF-κB inhibitor is incorporated into cigarette paper.

5. A method according to claim 3 wherein the NF-κB inhibitor is incorporated into tobacco filler.

6. A method according to claim 3 wherein the NF-κB inhibitor is incorporated as a separate ingredient into a tobacco containing product.

7. A method according to claim 3 wherein the NF-κB inhibitor is Tanshinone IIA or a diterpene derivative thereof which is a NF-κB inhibitor.

8. A method according to claim 3, wherein the NF-κB inhibitor is a standardized plant extract containing a naturally occurring NF-κB inhibitor.

9. A method according to claim 3, wherein the NF-κB inhibitor is selected from the group consisting of resveratrol, lactacystin, epigallocatechin, curcumin, pyrrolidine dithiocarbamate, herbamycin A, selenium, deguelin, idoxifene, raloxifene, droloxifene, tiremifene, and tamoxifen.
